(19) United States
(12) Patent Application Publication
(10) Pub. No.: US 2014/0257204 A1
(43) Pub. Date: Sep. 11, 2014

(54) APPARATUS FOR RECONSTITUTING AND DISPENSING DRUGS FOR TOPICAL APPLICATION

(71) Applicant: Stuart Robert Lessin, Haverford, PA (US)

(72) Inventor: Stuart Robert Lessin, Haverford, PA (US)

(21) Appl. No.: 13/786,392

(22) Filed: Mar. 5, 2013

(51) Int. Cl. A61M 35/00

(52) U.S. Cl. CPC A61M 35/003
USPC 604/290; 604/311

(57) ABSTRACT

Apparatus and methods for reconstituting and/or removing lyophilized and/or vialed drugs for topical application includes a syringe having a removable plunger. A vial extraction adapter can interconnect the syringe and a vial, allowing a user to transfer a reconstituting solution from the syringe to the vial. After appropriate mixing, the user can then easily remove the reconstituted drug solution from the vial into the syringe. A cap can then be placed on that end of the syringe and the plunger of the syringe can be removed. An application adapter can be placed over the opening from which the plunger was removed. A cap of the application adapter can cover an applicator end that can be used to apply the liquid drug substance from the syringe. The cap of the application adapter can be replaced and any unused drug produced can be stored until needed again.

### BACKGROUND OF THE INVENTION

The present invention relates to applicators and, more particularly, to an apparatus for reconstituting and dispensing lyophilized and/or vialed drugs for topical application.

Drugs manufactured for systemic use (intravenous or injection) can be used topically on the skin. There is no simple means of removing these drugs from their containers (vials) and applying them to the skin. Current methods to use systemic drugs for topical application require multiple steps and devices by an individual or reliance on a compounding pharmacist.

When an individual attempts to create a topically applied drug, there are often variations in the individual methods of removing drug from the vial, the methods of mixing the contents, the vessels used and the application methods. These variations can contribute to poor quality control of topical preparations and drug efficacy.

Some conventional devices and methods for preparing topical applications for lyophilized and/or vialed drugs are cumbersome and can expose the drug to contamination risks. Moreover, some conventional devices and methods can pose a potential unwanted drug exposure risk to individuals.

As can be seen, there is a need for an apparatus and methods for reconstituting and dispensing lyophilized and/or vialed drugs for topical application.

### SUMMARY OF THE INVENTION

In one aspect of the present invention, an apparatus for topical administration of a therapeutic agent comprises a syringe; a plunger fitting into a first end of the syringe and pushing contents of the syringe out a second end of the syringe; a vial extraction adapter fitting on the second end of the syringe; a needle end of the vial extraction adapter fitting into a vial; an applicator adapter fluidly connecting to the first end of the syringe when the plunger is removed therefrom; and an applicator end of the applicator adapter operable to deliver the therapeutic agent topically to a user.

In another aspect of the present invention, an apparatus for topical administration of a therapeutic agent comprises a syringe; a plunger fitting into a first end of the syringe and pushing contents of the syringe out a second end of the syringe; a vial extraction adapter fitting on the second end of the syringe; a needle end of the vial extraction adapter fitting into a vial; an applicator adapter fluidly connecting to the first end of the syringe when the plunger is removed therefrom; an applicator end of the applicator adapter operable to deliver the therapeutic agent topically to a user; a syringe cap removably fitting on the second end of the syringe to prevent fluid from escaping therefrom; and an applicator end cap removably fitting on the applicator adapter to cover the applicator end.

In a further aspect of the present invention, a method for topically applying a therapeutic agent comprises disposing a reconstituting solution in a syringe, the syringe having a plunger disposed in a first end thereof; fitting a vial extraction adapter to a second end of the syringe; attaching a vial to the vial extraction adapter; depressing the plunger to introduce the reconstituting solution into the vial; withdrawing the plunger to withdraw reconstituted therapeutic agent solution from the vial; removing the vial extraction adapter from the syringe; placing a syringe cap on the syringe to retain the reconstituted therapeutic agent in the syringe; removing the plunger from the syringe; placing an applicator adapter on the first end of the syringe; and delivering the reconstituted therapeutic agent in the syringe through the applicator adapter to an applicator end thereof.

These and other features, aspects and advantages of the present invention will become better understood with reference to the following drawings, description and claims.

### BRIEF DESCRIPTION OF THE DRAWINGS

FIG. 1 is a perspective view of various components used in the apparatus and methods for reconstituting and dispensing drugs for topical application, according to an exemplary embodiment of the present invention;

FIG. 2 is a side view of a syringe having reconstituting solution disposed therein;

FIG. 3 is a side view of the syringe of FIG. 2, having a vial extraction adapter attached thereto, illustrating attachment of a vial to the vial extraction adapter;

FIG. 4 is a side view of the syringe of FIG. 3 showing the vial attached to the vial extraction adapter and the reconstituting solution injected into the vial;

FIG. 5 is a side view of the syringe of FIG. 4 showing reconstituted drug solution being taken up in the syringe from the vial;

FIG. 6 is a partially cut-away side view of the syringe of FIG. 5 illustrating a cap being placed on the end of the syringe having the reconstituted drug solution disposed therein;

FIG. 7 is a side view of the syringe of FIG. 6, illustrating removal of a plunger therefrom;

FIG. 8 is a side view of the syringe of FIG. 7, with the plunger removed, illustrating placement of a base adapter on the end of the syringe from which the plunger was removed; and

FIG. 9 is a side view of the syringe of FIG. 8, illustrating removal of a cap of the base adapter to reveal an applicator end of the application adapter.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides an apparatus and methods for reconstituting and/or removing lyophilized and/or vialed drugs for topical application. The apparatus includes a syringe having a removable plunger. A vial extraction adapter can interconnect the syringe and a vial, allowing a user to transfer a reconstituting solution from the syringe to the vial. After appropriate mixing, the user can then easily remove the reconstituted drug solution from the vial into the syringe. A cap can then be placed on that end of the syringe and the plunger of the syringe can be removed. An application adapter can be placed over the opening from which the plunger was removed. A cap of the application adapter can cover an applicator end that can be used to apply the liquid drug substance from the syringe. The cap of the application adapter can be replaced and any unused drug produced can be stored until needed again.

The apparatus and methods of the present invention simplified and standardizes the method for reconstituting and dispensing lyophilized drugs from vials. The apparatus and methods of the present invention further reduce risks of contamination while enhancing ease of application.

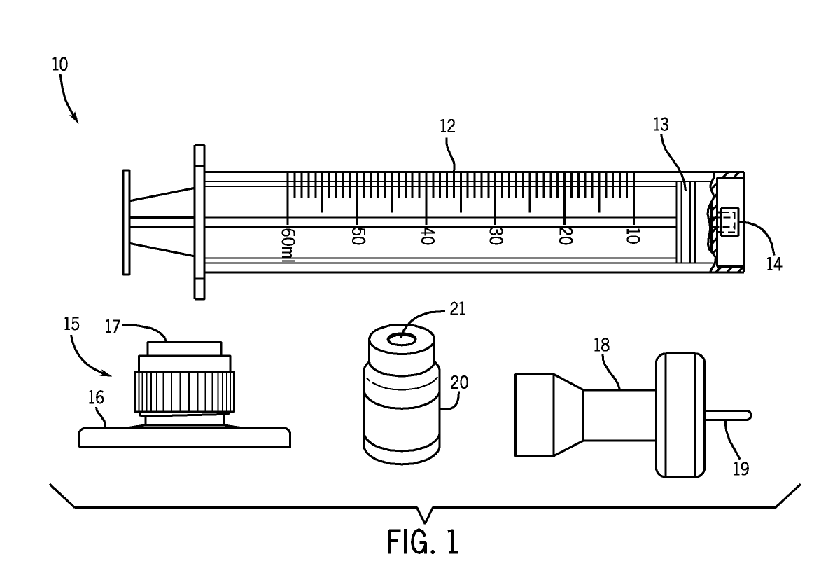

Referring to FIG. 1, a kit 10 for reconstituting and dispensing drugs for topical application includes a syringe 12 having a plunger 13 fitting therein. The syringe 12 can be a conventional disposable plastic syringe or may be a syringe designed for the purpose of the present invention. While the Figure shows a 60 ml syringe, other syringe volumes can be used within the scope of the present invention. The syringe 12 can have a first end, into which the plunger 13 is inserted, and a second end having a syringe cap 14 removably disposed thereupon. In some embodiments, the syringe cap 14 can sit within a recess so that when the syringe cap 14 is in place on the syringe 12 (as shown in FIG. 1), the syringe 12 can sit upright on the end having the syringe cap 14.

The kit 10 can include a vial extraction adapter 18 that interconnects the syringe 12 with a vial 21. A needle end 19 of the vial extraction adapter 18 can puncture a membrane 21 of the vial 20 to access the contents within the vial 20.

The kit 10 can include an applicator adapter 15. A base 16 of the applicator adapter 15 can attach to the first end of the syringe 12 when the plunger 13 is removed. The applicator adapter 15 can allow liquid in the syringe 12 to flow to an applicator end 26 (see FIG. 9) of the applicator adapter 15. A cap 17 can removably cover the applicator end 26 of the applicator adapter 15.

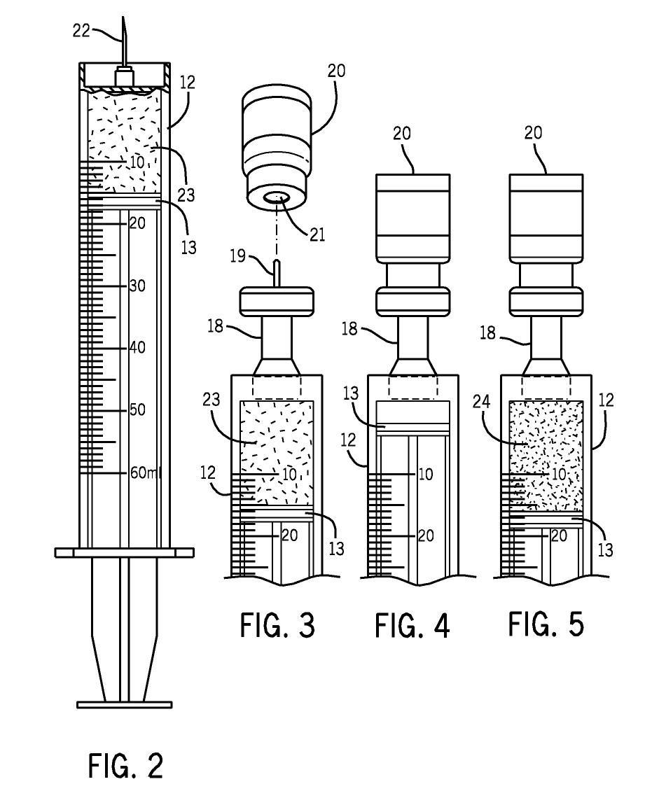

Referring now to FIGS. 2 through 9, exemplary steps for using the kit 10 of the present invention are pictorially shown. A syringe needle 22 can be removable attached to the syringe 12 upon removal of the cap 14. A reconstituting solution 23 can be drawn up into the syringe 12 via the needle 22 via conventional processes. In some embodiments, the syringe 12 can be supplied with the reconstituting solution 23 predisposed therein. FIG. 2 shows the reconstituting solution 23 in the syringe 12.

The vial extraction adapter 18 can be placed on the syringe 12 and a vial 20 can be attached to the needle end 19 of the vial extraction adapter 18, as shown in FIG. 3. In this configuration (shown interconnected in FIG. 4), there is a fluid communication between the reconstituting solution 23 and the inside of the vial 20. In some embodiments the needle 22 can be used to pass into the vial extraction adapter. In other embodiments, the needle 22 can be removed and the end of the syringe 12 can simply engage with the vial extraction adapter 18 to form a fluid connection therebetween.

The plunger 13 can be depressed to introduce the reconstituting solution 23 into the vial, as shown in FIG. 4. The reconstituting solution 23 can be mixed accordingly inside the vial 20, as may be required to dissolve, place in solution, or place in suspension the drug product in the vial 20.

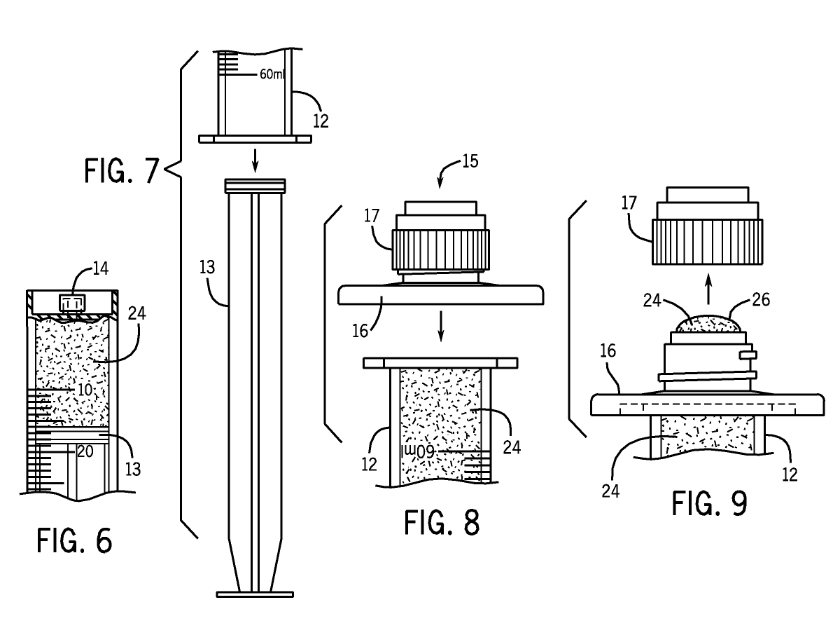

As shown in FIG. 5, the user can then draw reconstituted drug solution 24 into the syringe 12. The vial extraction adapter 18 can be removed from the syringe 14 and the cap 14 can be placed on the end of the syringe 12 to fluidly retain the reconstituted drug solution 24 within the syringe 12, as shown in FIG. 6.

The plunger 13 can be removed, as shown in FIG. 7, from the syringe 12 and the base 16 of the application adapter 15 can attach to the end of the syringe 12 from which the plunger 13 was withdrawn, as shown in FIG. 8. The base 16 can make a fluidly sealed connection with the syringe 12 such that the reconstituted drug solution 24 can pass into the application adapter 15 and onto the applicator end 26 of the application adapter 15. The applicator end 26 can be designed in various manners, such as being an absorbent sponge-like material, a roller ball, or the like, to allow the reconstituted drug solution 24 to be applied topically. A cap 17 can be removably placed over the applicator end 26. The cap 17 may help protect the applicator end 26 and prevent unintentional application of the reconstituted drug solution 24 when the syringe 12 is not in use.

The kit 10 of the present invention can be used for topical delivery of various drug products. These drug products, disposed in the vial 20, can be liquid drug products, solid (lyophilized) drug products, or solutions of drug products. The reconstituting solution 23 can be various compositions. Typically, the reconstituting solution 23 can be formed to allow the drug product in the vial 20 to dissolve therein. In some embodiments, the reconstituting solution 23 can include ingredients designed to assisted in the transdermal delivery of drug product.

It should be understood, of course, that the foregoing relates to exemplary embodiments of the invention and that modifications may be made without departing from the spirit and scope of the invention as set forth in the following claims.

What is claimed is:

1. An apparatus for topical administration of a therapeutic agent, the apparatus comprising:
a syringe;
a plunger fitting into a first end of the syringe and pushing contents of the syringe out a second end of the syringe;
a vial extraction adapter fitting on the second end of the syringe;
a needle end of the vial extraction adapter fitting into a vial;
an applicator adapter fluidly connecting to the first end of the syringe when the plunger is removed therefrom; and
an applicator end of the applicator adapter operable to deliver the therapeutic agent topically to a user.

2. The apparatus of claim 1, further comprising a syringe cap removably fitting on the second end of the syringe to prevent fluid from escaping therefrom.

3. The apparatus of claim 1, further comprising an applicator end cap removably fitting on the applicator adapter to cover the applicator end.

4. The apparatus of claim 2, wherein the syringe cap is recessed in the second end of the syringe.

5. An apparatus for topical administration of a therapeutic agent, the apparatus comprising:
a syringe;
a plunger fitting into a first end of the syringe and pushing contents of the syringe out a second end of the syringe;
a vial extraction adapter fitting on the second end of the syringe;
a needle end of the vial extraction adapter fitting into a vial;
an applicator adapter fluidly connecting to the first end of the syringe when the plunger is removed therefrom;
an applicator end of the applicator adapter operable to deliver the therapeutic agent topically to a user;
a syringe cap removably fitting on the second end of the syringe to prevent fluid from escaping therefrom; and
an applicator end cap removably fitting on the applicator adapter to cover the applicator end.

6. A method for topically applying a therapeutic agent, comprising:
disposing a reconstituting solution in a syringe, the syringe having a plunger disposed in a first end thereof;
fitting a vial extraction adapter to a second end of the syringe;
attaching a vial to the vial extraction adapter;
depressing the plunger to introduce the reconstituting solution into the vial;
withdrawing the plunger to withdraw reconstituted therapeutic agent solution from the vial;
removing the vial extraction adapter from the syringe;
placing a syringe cap on the syringe to retain the reconstituted therapeutic agent in the syringe;
removing the plunger from the syringe;
placing an applicator adapter on the first end of the syringe; and
delivering the reconstituted therapeutic agent in the syringe through the applicator adapter to an applicator end thereof.

7. The method of claim 6, further comprising penetrating the vial with a needle end of the vial extraction adapter.

8. The method of claim 6, further comprising removably fitting an applicator end cap on the applicator adapter to cover the applicator end.

9. The method of claim 6, wherein the syringe cap is recessed in the second end of the syringe.
